(19) United States
(12) Patent Application Publication
(10) Pub. No.: US 2011/0202109 A1
(43) Pub. Date: Aug. 18, 2011

(54) VISION REGENERATION ASSISTING APPARATUS

(75) Inventors: Motohiro KAMEI, Minoo-shi (JP); Hirokazu SAKAGUCHI, Minoo-shi (JP); Yasuo TERASAWA, Obu-shi (JP); Masayuki SHINOMIYA, Gamagori-shi (JP)

(73) Assignee: NIDEK CO., LTD., Aichi (JP)

(21) Appl. No.: 13/027,392

(22) Filed: Feb. 15, 2011

(30) Foreign Application Priority Data
Feb. 16, 2010 (JP) P.2010-031671

(51) Int. Cl. A61F 9/08

(52) U.S. Cl. 607/53

(57) ABSTRACT

A vision regeneration assisting apparatus for regenerating a patient's vision, includes: a housing; a plurality of needle-like electrodes that are formed at the housing and extend from the housing to a predetermined length, the electrodes being configured to stick into an optic papilla of a patient's eye; and a needle-like fixing portion that is formed at the housing, is separated from the electrodes, is formed so that the fixing portion extends from the housing in the same direction in which the electrodes extend, and is configured to maintain a state where the electrodes are stuck in the optic papilla.

### BACKGROUND

The disclosure relates to a vision regeneration assisting apparatus providing a visual signal to a patient artificially.

As a technique for blindness treatment, there has been studied a vision regeneration assisting apparatus which promotes vision regeneration by electrically stimulating cells and optic nerve fibers constituting the retina with an intraocular device implanted within the eyes. As the vision regeneration assisting apparatus, there has been devised an optic nerve stimulating device in which a plurality of needle-like electrodes are inserted into the optic papilla to implant the tip of device and the electrodes output electric stimulation pulse signals to stimulate the optic nerve fiber so as to provide vision to a patient (JP-A-2004-181100 (US 2006058857)).

In the optic nerve stimulating device, since the plurality of electrodes are inserted into the optic disk one by one, operative time is lengthened, and which is thus laborious for both the patient and surgeon. Moreover, there is a possibility that the electrodes inserted into the optic papilla and wires (signal wires) tying up the electrodes will come off due to eye movement after implantation, and be broken due to metal fatigue.

One aspect of the exemplary embodiment is to provide a vision regeneration assisting apparatus in which a plurality of electrodes can be efficiently attached to the optic papilla, and which can be stably handled for a long time.

### SUMMARY

In order to achieve the above object, the invention includes the following configuration.

(1) A vision regeneration assisting apparatus for regenerating a patient's vision, comprising:

a housing;

a plurality of needle-like electrodes that are formed at the housing and extend from the housing to a predetermined length, the electrodes being configured to stick into an optic papilla of a patient's eye; and

a needle-like fixing portion that is formed at the housing, is separated from the electrodes, is formed so that the fixing portion extends from the housing in the same direction in which the electrodes extend, and is configured to maintain a state where the electrodes are stuck in the optic papilla.

(2) The vision regeneration assisting apparatus according to (1), wherein a part of the fixing portion extending from the housing is longer that parts of the electrodes extending from the housing.
(3) The vision regeneration assisting apparatus according to (2) comprising a gripping portion for gripping the housing.
(4) The vision regeneration assisting apparatus according to (3), wherein the fixing portion and the gripping portion are molded integrally to be a single member.
(5) A vision regeneration assisting apparatus for regenerating a patient's vision, comprising:

a plurality of electrodes configured to stick into an optic papilla of a patient's eye;

a controller configured to control the plurality of electrodes to output an electric stimulation pulse signal, respectively; and

a connecting member electrically connecting the electrodes to the controller,

wherein the connecting member includes:

a plurality of first signal wires, each of which is a single wire connected to each of the electrodes;

a plurality of second signal wires, each of which is a stranded wire and includes one end connected to each of the first signal wire and the other end connected to the controller; and

a connecting portion connecting the first signal wire to the second signal wire outside the eyeball.

(6) The vision regeneration assisting apparatus according to (5), wherein the plurality of second signal wires are bundled and wound in a spiral to form one cable.
(7) The vision regeneration assisting apparatus according to (6), wherein the first signal wire has a curve shape curved with a radius of curvature of 10 mm or longer and 20 mm or shorter.
(8) The vision regeneration assisting apparatus according to (7), wherein

the connecting portion is made of a metal pipe,

the first signal wire is welded to and is connected to the second signal wire, and

the first and second signal wires are inserted in the pipe.

### BRIEF DESCRIPTION OF THE DRAWINGS

FIG. 1 is a view illustrating a use embodiment of a vision regeneration assisting apparatus.

FIG. 2 is a block diagram illustrating the schematic configuration of a vision regeneration assisting apparatus.

FIG. 3 is a block diagram illustrating the configuration of an image processing device.

FIG. 4 is a schematic configuration view of a stimulation condition setting unit.

FIGS. 5A and 5B are configuration views of a connecting member and a stimulating unit.

FIGS. 6A and 6B are a schematic diagram illustrating the vicinity of connecting member and a view illustrating an internal configuration.

FIG. 7 is a view schematically illustrating the implantation state of electrodes.

FIG. 8 is a view illustrating the positional change of phosphene according to the change of stimulating signal condition.

FIGS. 9A and 9B are views schematically illustrating a second embodiment of an electrode portion.

### DESCRIPTION OF EXEMPLARY EMBODIMENTS

An exemplary embodiment will be described with reference to the drawings.

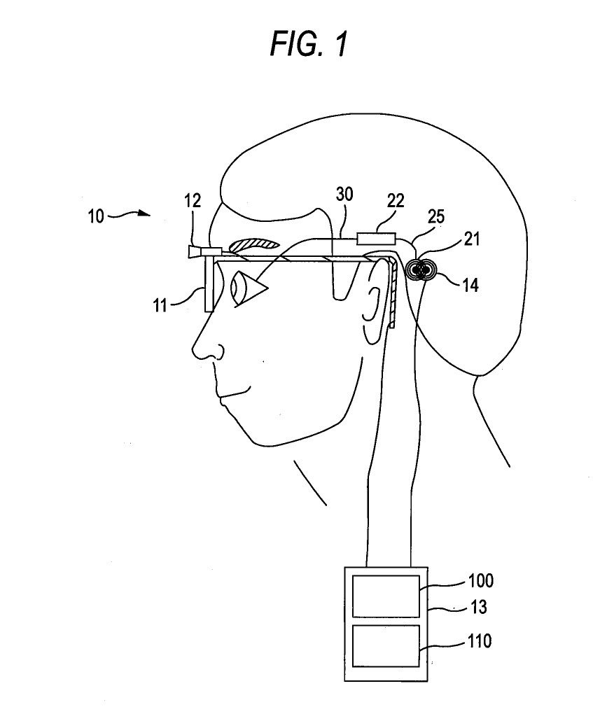

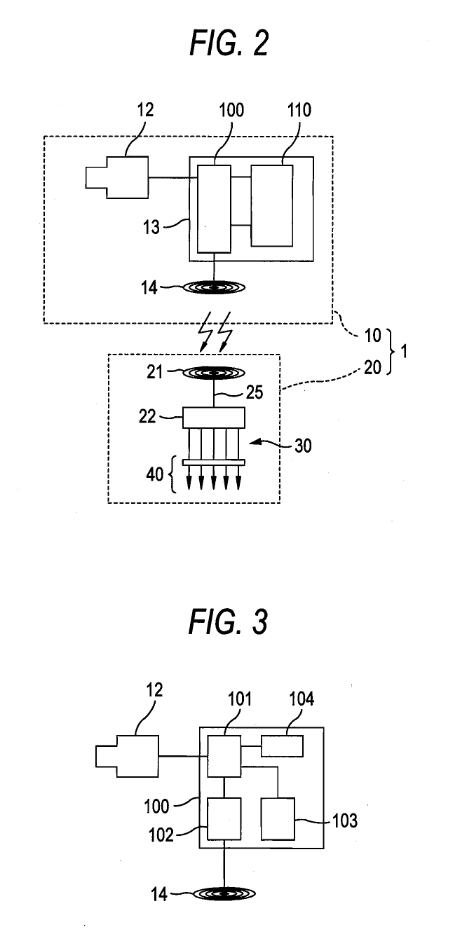

FIG. 1 is a view illustrating a use embodiment of a vision regeneration assisting apparatus. FIG. 2 is a block diagram of the vision regeneration assisting apparatus. FIG. 3 is a block diagram of an image processing device 100.

A vision regeneration assisting apparatus 1 includes an external body device 10 which a patient wears, and an internal body device 20 implanted to the patient by surgery. The external body device 10 includes a visor 11 which the patient wears, a photographing device 12 such as a CCD camera provided to the visor 11, an external device 13, and a transmitter 14 including a primary coil. The visor 11 looks like eyeglasses, and is put in front of the patient's eyes (refer to FIG. 1). The photographing device 12 is provided on a front surface of the visor 11, and takes a picture of a subject to be perceived by the patient. The photographing device 12 may be provided in such a state that it can take pictures in the direction that the patient faces.

The external device 13 includes an image processing device 100 and a battery 110. The image processing device 100 performs an image processing on the subject image taken by the photographing device 12, and generates data for electric stimulation pulse for generating vision. The battery 110 supplies power to the entire vision regeneration assisting apparatus 1 (including the external body device 10 and the internal body device 20).

The image processing device 100 includes a controller 101, a pulse signal converter 102, a stimulation condition setting unit 103, and a storage unit 104. The controller 101 includes a CPU or the like and controls the driving of entire external device 13. The pulse signal converter 102 performs an image processing on the subject image taken by the photographing device 12, and generates the data for an electric stimulation pulse. The pulse signal converter 102 transmits the data for the electric stimulation pulse to the internal body device 20 via the transmitter 14. The stimulation condition setting unit 103 includes an adjustment dial, various switches, and the like. The stimulation condition setting unit 103 can set the output condition of electric stimulation pulse signal output from a stimulating electrode portion (referred to as an electrode portion hereinbelow) 41 of the internal body device 20, according to the generation position and the shape of simulated vision called phosphene (light reception, flash) perceived by the patient. The controller 101 of image processing device 100 is connected with the photographing device 12, the pulse signal converter 102, the stimulation condition setting unit 103, and the storage unit 104.

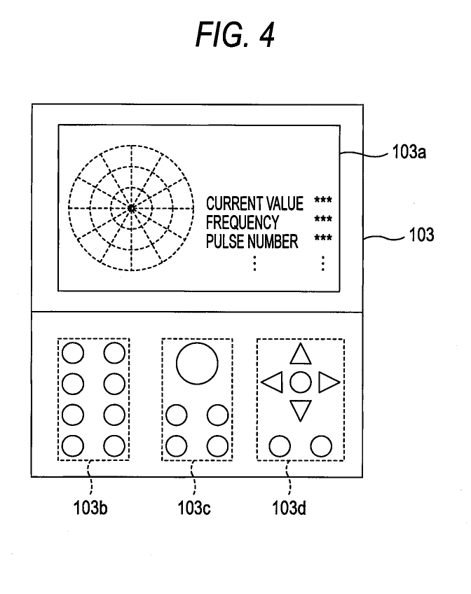

FIG. 4 is a view illustrating the configuration of the stimulation condition setting unit 103. A display unit 103a displays various setting conditions and the generation position of phosphene. An output electrode assigning unit 103b is provided with switches for assigning the electrode which outputs the electric stimulation pulse signal. An output condition setting unit 103c is provided with switches and an adjustment dial for setting the output condition of electric stimulation pulse signal. A phosphene position setting unit 103d is provided with switches for recording at which position the phosphene is generated within the patient's visual field by the electric stimulation pulse signal output from the electrode (stimulation condition set by the stimulation condition setting unit 103 will be described later in detail.). The storage unit 104 stores a plurality of stimulation conditions set by the stimulation condition setting unit 103 and the corresponding phosphenes while being correlated each other.

It is preferable for the external device 13 to be large enough to be carried by the patient. As two completely separate devices, the stimulation condition setting unit 103 and the external device 13 can be connected to each other as necessary through any communication device (connection device) such as USB. In this case, the condition set by the stimulation condition setting unit 103 is stored in the storage unit 104. The stimulation condition setting unit 103 can be detached upon use, whereby the external device 13 can be more compact.

The transmitter 14 including a primary coil transmits (wireless transmission) the data for the electric stimulation pulse signal and power to a receiver 21 (a secondary coil) of the internal body device 20 as electromagnetic waves. A magnet (not shown in the figure) is provided in the center of transmitter 14. The magnet fixes the position of receiver 21 and improves the data transmission efficiency of the transmitter 14. The present embodiment shows a case where the wireless transmission is performed by electromagnetic induction, but it is also possible to perform the wire transmission by directly connecting the external body device 10 with the internal body device 20.

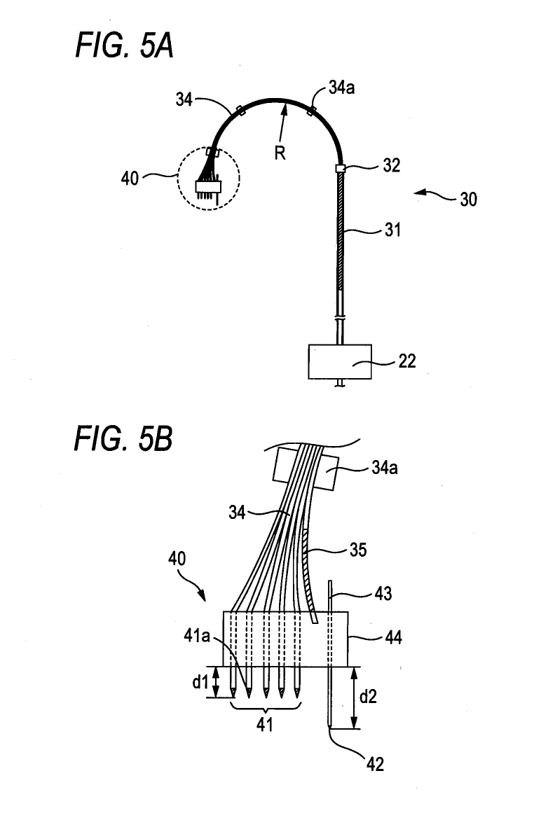

The internal body device 20 includes a receiver 21 including a secondary coil, a signal wire 25, an information processing control unit 22, a connecting member 30, and a stimulating unit 40. The receiver 21 and the information processing control unit 22 are connected with each other through the signal wire 25, and disposed outside the eyeball. On the other hand, the stimulating unit 40 is electrically connected with the information processing control unit 22 through the connecting member 30, and disposed inside the eyeball. The connecting member 30 includes a cable 31, a connecting portion 32, and signal wires 34. The cable 31, the connecting portion 32, and a portion of the signal wires 34 are disposed outside the eyeball, and the remaining portion of the signal wires 34 is disposed inside the eyeball (refer to FIG. 5A).

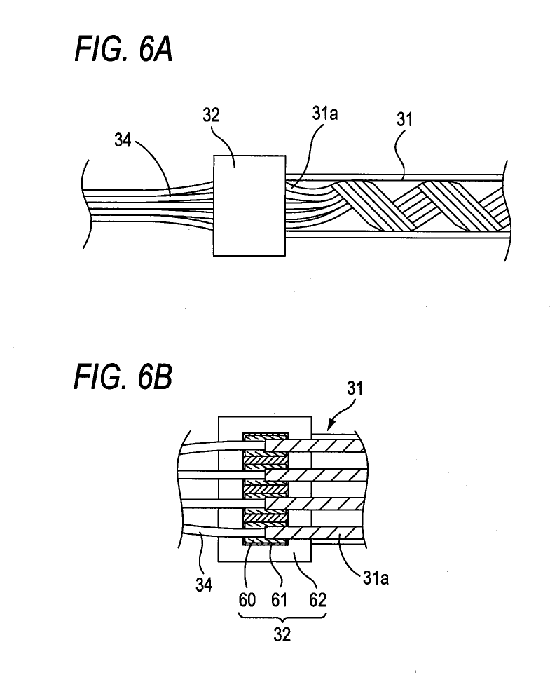

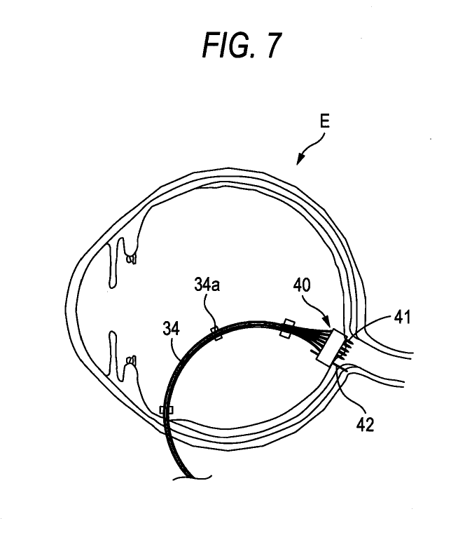

Herein, FIG. 5 illustrates the configuration of connecting member 30 and the stimulating unit 40. FIG. 5A is a view illustrating the configuration from the information processing control unit 22 and the stimulating unit 40, and FIG. 5B is a magnified view illustrating the vicinity of a stimulating unit 40. In FIG. 5A, one end of the cable 31 is connected to the information processing control unit 22, and the other end thereof is connected to the connecting portion 32 outside the eyeball. The signal wires 34 are connected to the connecting portion 32, and most of the signal wires 34 are placed in the eye. A portion of the end of the signal wires 34 is used as an electrode portion 41, and electrodes 41a are formed at the end of electrode portion 41 (signal wires 34) (the electrode portion 41 will be described later in detail). In this configuration, the signal is transmitted from the information processing control unit 22 to the electrode portion 41. The cable 31 is formed by integrally tying and coiling up the signal wires 31a connected to the signal wires 34. (refer to FIG. 6). The respective signal wires 31a are formed into one wire made of a plurality of wires (single wires) of metal such as platinum.

The configuration of the stimulating unit 40 will now be described in detail. As shown in FIG. 5B, the stimulating unit 40 includes a needle-like electrode portion 41 having a plurality of electrodes 41a formed at the end thereof, a needle-like fixing portion 42 for fixing the position of the electrode portion 41 inserted into the optic papilla, a housing 44 where the electrode portion 41 and the fixing portion 42 are provided in an integrated manner, and a gripping portion 43 used for a surgeon to grip the housing 44 with a pair of forceps or the like when the surgeon installs the stimulating unit 40 in the optic papilla. The electrode portion 41 includes a portion of the end of signal wires 34, and is long enough to be inserted into the optic papilla and to provide the electric stimulation. In other words, the electrode portion 41 is constituted with the end of signal wires 34 which penetrates through the housing 44 and is extended to a predetermined length. The electrodes 41a are formed at the end of the electrode portion 41. The electrodes 41a are formed by removing a portion of insulating coating film of the electrode portion 41 (the end of signal wires 34). Although five electrodes 41a are formed in the embodiment, the number of electrodes 41a may be at least four or more. In addition, in this embodiment, there is provided a signal wire 35 which does not form the electrodes 41a at its end and is used as a reference electrode (a counter electrode). The reference electrode is obtained by removing a portion of coating on the periphery of signal wire 35.

As the insulating coating film of signal wires 34 (electrode portion 41) described above, materials having an insulation property and excellent biocompatibility such as Teflon (a registered trademark), polyimide, and polyparaxylylene are used. For the electrodes 41a (conductive wires of the signal wires 34), an alloy of platinum (Pt) and iridium (Ir) having biocompatibility is used. In the embodiment, an alloy of platinum (90%) and iridium (10%) is used. In addition, as the material of electrodes 41a, conductive materials which have such a degree of rigidity that the material can be inserted into the optic papilla, and are excellent in the biocompatibility and durability are selected. For instance, platinum, iridium oxide, and stainless steel having biocompatibility can be used.

The electrodes 41a have a shape of a needle that can be easily inserted into the optic papilla, and the end of electrodes 41a is so formed that it does not easily come off from the optic papilla after insertion. The diameter (thickness) of the electrodes 41a is so determined that the electrodes 41a have the rigidity of an extent at which they are not bent accidentally when they are inserted into the optic papilla. For example, the electrodes 41a are formed to have a diameter of 50 μm in the embodiment. The electrodes 41a can also be formed to have a diameter of 20 μm or larger and 100 μm or smaller. If the diameter of electrodes 41a is smaller than 20 μm, the electrodes 41a are prone to bend due to buckling when they are inserted into the optic papilla. On the other hand, if the diameter of electrodes 41a is larger than 100 μm, the proportion of electrodes 41a to the optic papilla is not local, which is likely to lead to nerve atrophy or the like.

The diameter of electrodes 41a may be determined to satisfy the above described property according to the property of the electrode materials. However, the larger the diameter of electrodes 41a, the smaller the number of electrodes 41a which can be inserted into the optic papilla. Therefore, it is preferable for the electrodes 41a to be formed to be as fine as possible. A length d1 of the electrode portion 41 (the length extended from the housing 44) is formed to be 0.5 mm or longer and 2 mm or shorter for example. If the length d1 of electrode portion 41 is shorter than 0.5 mm, it is difficult for the electrode portion 41 to reach the depth necessary for signal transmission when the electrode portion 41 is provided to the optic papilla. On the other hand, if the length d1 is longer than 2 mm, the electrode portion 41 is prone to bend due to buckling when it is inserted into the optic papilla. In the embodiment, the length d1 of electrode portion 41 is 1 mm for example.

In order to locally stimulate the optic nerve, it is preferable that the area of electrodes 41a is as small as possible. Therefore, in the embodiment, only a portion of the end of electrode portion 41 having a predetermined length d1 is used as the electrodes 41a. In this manner, regardless of the length d1 of electrode portion 41, it is possible to provide the electrodes 41a with a predetermined area which is suitable for locally stimulating the optic nerve. For instance, in the embodiment, the electrodes 41a are formed by removing about 0.5 mm of the coating film of the signal wires 34 constituting the electrode portion 41, from the distal end to the proximal end. It is also possible to use the entire electrode portion 41 as the electrodes 41a.

The fixing portion 42 is formed of materials which have rigidity and are excellent in the biocompatibility and durability. For example, an alloy of platinum (80%) and iridium (20%) is used for the fixing portion 42. The fixing portion 42 may also be formed of ceramics or hard resin. The fixing portion 42 is formed into a needle-like member extending to a predetermined length, in the same direction as the electrode portion 41 (electrodes 41a) provided in the housing 44. At this time, it is preferable for the fixing portion 42 to be formed to be longer than the electrodes 41a. This configuration makes it difficult for the electrodes 41a inserted into the optic papilla to come off later due to the eye movement. For instance, in the embodiment, a length d2 of the fixing portion 42 is 3 mm, which is sufficiently longer than the electrodes 41a. The length d2 of fixing portion 42 may be formed to be 0.5 mm or longer and 4 mm or shorter. If the lengths of the fixing portion 42 and the electrodes 41a are almost the same, it is preferable to perform a slipping-out prevention treatment by providing plural wedges to the fixing portion 42 for example. The diameter (thickness) of the fixing portion 42 may be set to such a size so as to provide rigidity at which the fixing portion 42 does not bend when it is inserted into the optic papilla and that hardly compresses the nerve. For example, the diameter is formed to be 50 μm or larger and 200 μm or smaller. Herein, the diameter is formed to be about 100 which is twice the thickness of the electrodes 41a.

The fixing portion 42 may be used as the reference electrode. In this case, the fixing portion 42 is formed of conductive materials so as to have the above properties and connected to the information processing control unit 22.

The gripping portion 43 is formed into such a shape that the surgeon can perform gripping with a pair of forceps when the stimulating unit 40 is provided to the optic papilla. In FIG. 5, the fixing portion 42 and the gripping portion 43 are integrally molded into a stick shape, and connected to each other to penetrate through the housing 44. In this configuration, the distal end thereof is used as the fixing portion 42, and the proximal end thereof is used as the gripping portion 43. The fixing portion 42 and the gripping portion 43 may also be formed separately. For example, the gripping portion 43 may be formed in the lateral position of the housing. The gripping portion 43 can also be formed in a position where the gripping portion 43 can be easily held by the surgeon's forceps. When the housing 44 has a shape which can be easily held, the gripping portion 43 need not be provided in the stimulating unit 40. In this case, the surgeon directly holds the housing 44 with a pair of forceps for example. In this manner, the stimulating unit 40 is installed to the optic papilla while the surgeon holds the gripping portion 43 (or the housing 44 itself).

The housing 44 is formed of materials, such as silicon, which have biocompatibility and hardness by which the shape of the housing 44 can be maintained in a body. The shape of the housing 44 is formed by a molding process for example. During the molding process, a plurality of through holes are formed in the thickness direction of housing 44 in correspondence with the position through which the signal wires 34 (electrodes 41a) are provided. The signal wires 34 (electrodes 41a) are then provided through the through holes, and the position through which the signal wires 34 (electrodes 41a) are provided is fixed by being solidified with silicon resin for example.

It is preferable for the size of housing 44 to be as small as the diameter of the optic papilla. With this configuration, the electrodes 41a fixed with the housing 44 are uniformly provided to the optic papilla. If the size of the housing 44 is formed to be as small as possible so that the electrodes 41a can be disposed in the optic papilla, it is possible to reduce the size of the incision made to the eyeball, whereby the housing 44 (stimulating unit 40) can be easily provided in the eye.

It is preferable for the fixing portion 42 to be provided near the periphery of the housing 44. In this manner, in a state where the electrodes 41a are provided in the central portion of the optic papilla, the fixing portion 42 is inserted into the periphery of the optic papilla which is hardly affected. The plurality of signal wires 34 heading to the cable 31 from the housing 44 are properly bundled together by a tube 34a in the middle of the wires, whereby the signal wires 34 can be easily handled. As the tube 34a, a tube made of materials, such as silicon, which are excellent in biocompatibility and flexibility is used. It is also possible to promote easy workability of the plurality of signal wires 34 by solidifying the signal wires 34 with the resin having biocompatibility.

The signal wires 34 are formed into a curve shape with a predetermined curvature. In the embodiment, the signal wires 34 connecting the housing 44 to the cable 31 are formed into a curve shape with a predetermined radius of curvature of 10 mm or longer and 20 mm or shorter. This configuration makes it possible to push in the stimulating unit 40, which is put into the eye through the vicinity of the corneal limbus of the eyeball, into the optic papilla without bending on the way, whereby it becomes easier to insert the electrode portion 41 and the like. The curve of the signal wires 34 is formed by firmly winding the material (wire rod) of the signal wires 34 around a cylindrical member having a predetermined curvature in advance.

It is preferable for the length of signal wires 34 to be 30 mm or longer and 38 mm or shorter in a linear distance from the end of electrode portion 41. In this manner, the connecting portion 32 for connecting the signal wires 34 to the cable 31 is properly placed outside the eyeball (if the connecting portion 32 is provided in a position far from the eyeball, the surgery becomes difficult). The length of the signal wires 34 may be determined according to the patient's eyeball size.

The cable 31 connected to the information processing control unit 22 forms a stranded wire (signal wires 31a) which is a combination of a plurality of single wires (diameter of 25 μm for example) formed of the alloy of platinum and iridium. The insulation coating is performed on the outside of the stranded wire with parylene or the like, and the resultant is buried (embedded) in insulating materials, such as silicon, having biocompatibility and flexibility. The plurality of signal wires 31a is entirely coated in this way, followed by a further coating with silicon or the like, whereby the signal wires 31a are integrated in a state where the respective wires are insulated separately. Since the outside of the signal wires 31a is formed of a flexible material, the load applied to the body is lessened.

In the cable 31 within a predetermined range from the connecting portion 32 (220 mm for example), the plurality of signal wires 31a are wound in a spiral as described above. When the eye is moved in the state where the stimulating unit 40 is provided in the optic papilla, load is applied to the cable 31. If the load is applied for a long time, the inside of the cable 31 is deteriorated, and as a result, the signal wires 34 are prone to be broken. Therefore, by making the signal wires 31a into the stranded wire and forming the signal wires 31a within the range to which the load of cable 31 is applied (it is known that the range is a predetermined range from the connecting portion 32) into a spiral shape, the durability of the signal wires 31a is improved. In this manner, it is possible to stably use the internal body device 20 for a long time. In this embodiment, the signal wires 31a constituting a portion of range of the cable 31 are formed into a spiral shape; however, the signal wires 31a constituting the entire cable 31 may be formed into a spiral shape.

The cable 31 (signal wires 31a) having the above configuration is connected to the signal wires 34 through the connecting portion 32. FIG. 6A is a schematic diagram illustrating the vicinity of connecting portion 32, and FIG. 6B is a diagram illustrating the internal structure of the connecting portion 32. In the connecting portion 32, signal wires 34 and the corresponding signal wires 31a of the cable 31 are respectively connected to each other through pipes 60 formed into a hollow shape. As the pipe 60, a pipe made of metal having excellent biocompatibility such as platinum is used. While the tip of the signal wires 34 and the tip of the signal wires 31a are put into the pipes 60, the signal wires of both parties are joined together by welding. Using the pipes 60 makes it simple to connect the signal wires 31a of the cable 31 and the signal wires 34, which are not easily connected to each other.

The respective pipes 60 connecting the signal wires 34 to the signal wires 31a are solidified by a coating layer 61 made of epoxy resin having biocompatibility, with gaps enabling insulation to be maintained. In this manner, since the signal wires 34 and the single wires 31a are respectively connected to each other without gaps, permeation of liquid or the like is properly prevented. The periphery of the coating layer 61 is solidified by the resin made of the material, such as silicon, having biocompatibility and flexibility, whereby an outer shape 62 of the connecting portion 32 is formed. Since the outside of the connecting portion 32 is made of the flexible material, the load applied in the body is lessened, and therefore, it is possible to properly position the connecting portion 32.

The internal body device 20 having the above configuration is implanted in the patient's body by surgery. The method of implanting the internal body device 20 will now be described. FIG. 7 is a cross sectional view entirely illustrating an eyeball E in which the stimulating unit 40 is provided in the optic papilla.

First, the surgeon grips the gripping portion 43 by using a pair of forceps, inserts the stimulating unit 40 through the incision opening formed in the sclera of the patient's eye from which the crystalline lens is removed, and keep pushing in the stimulating unit 40. The surgeon then positions the stimulating unit 40 on the optic papilla while the surgeon is gripping the gripping portion 43, and sticks the electrode portion 41 in the optic papilla to a predetermined depth. At this time, the fixing portion 42 formed to be longer than the electrode portion 41 is inserted first in the periphery of the optic papilla. In this manner, the electrode portion 41 inserted into the position of the optic papilla is more firmly fixed. By the operation, the plurality of electrodes 41a are placed in the optic papilla in a single operation. In the embodiment, since the signal wires 34 have a shape which is curved along the inside of the eye, it is possible to easily push in the stimulating unit 40 to the vicinity of the optic papilla, whereby the surgeon's labor is lessened during the surgery. The tube 34a is fixed in the eye by tacks (not shown) or other fixing means such as sutures. If unnecessary, the tube 34a may be left unfixed.

The position of the connecting portion 32 positioned outside the eyeball is fixed near the eyeball by sutures for example. The transmitter 14 and the information processing control unit 22 are subcutaneously implanted at the position of the patient's head. At this time, the signal wires 34 (tube 34a) and 25 are also implanted (refer to FIG. 1). The transmitter 14 is placed at the position where the receiver 21 is implanted, in such a manner that the transmitter 14 is superimposed on the receiver 21 with the skin therebetween. In this manner, the external body device 10 and the internal body device 20 are held by magnetic force, and their position is fixed.

Now the vision regeneration operation performed by using the vision regeneration assisting apparatus including the above configuration will be described. It is known that when the electric stimulation is provided to the cells and optic nerves constituting the retina of the eye, the simulated vision called phosphene (light reception, flash) is produced. In the invention, vision regeneration is promoted by inserting electrodes in the optic papilla and causing the electrodes to output the predetermined electric stimulation pulse signal to make the patient perceive the phosphene.

As advance preparations, the surgeon checks the position where the phosphene is produced according to various stimulation conditions set by the stimulation condition setting unit 103 and which is specific to the patient. First, by the output electrode assigning unit 103b, it is set whether the electric stimulation pulse signal is output from one electrode or from a plurality of electrodes simultaneously. Next, by using the output condition setting unit 103c, the output condition of respective electrodes outputting the electric stimulation pulse signal is set. In the output condition setting unit 103c, output conditions such as the current value, the frequency, the pulse width, the charge amount, the waveform (monophasic waveform and two-phase waveform), the stimulation resting phase between pulses (interpulse), and the pulse number are set. The details of setting are displayed on the display unit 103a.

After the stimulation condition for making phosphene perception is set, a stimulation start switch (not shown) is pressed. In this manner, the electric stimulation pulse signal is output from the electrodes 41a according to the set stimulation condition, whereby the optic nerve is stimulated. After the output of the electric stimulation pulse signal, the operator (surgeon) asks the patient about how big the phosphene is and where the phosphene appears in the patient's visual field.

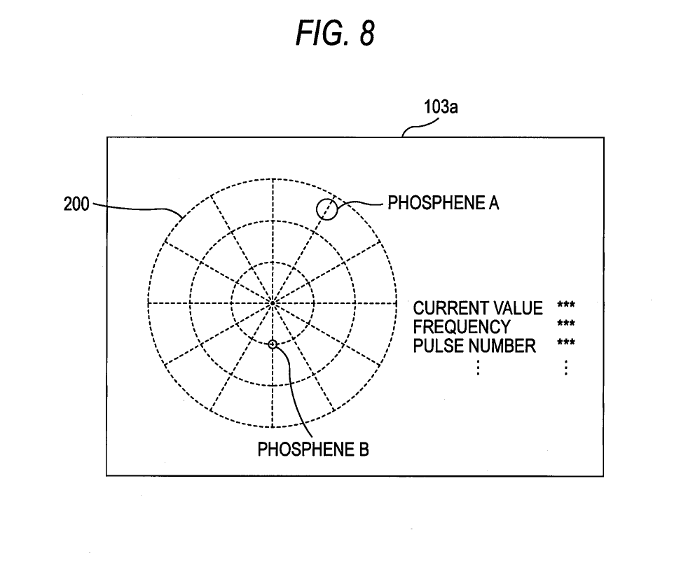

FIG. 8 is a view illustrating the positional change of phosphene according to the change of stimulating signal condition. The display unit 103a displays a chart 200 for displaying the position where the phosphene appears and the shape of the phosphene. By using the phosphene position setting unit 103d, the operator displays and records the position and the shape of the phosphene appearing on the chart 200.

In this manner, the stimulation is provided with bringing variety to the stimulation condition, until respective phosphenes are checked over the patient's entire visual field. The phosphene is mapped on the chart 200. After checking the generation position of the phosphene in the entire visual field, the surgeon operates a save switch (now shown), thereby saving the shape and the positional information of respective phosphene and the corresponding stimulation condition of the electric stimulation pulse signal in the storage unit 104 in the correlated manner.

After the completion of the advance preparations, the vision regeneration assisting apparatus 1 is operated. The imaging data (image information) of the subject taken by the photographing device 12 is transmitted to the image processing device 100. The controller 101 transmits the received image information, the shape and the positional information of the phosphene which is specific to the patient and stored in the storage unit 104 to the pulse signal converter 102. Based on the image information and the shape and the positional information of the phosphene, the pulse signal converter 102 extracts the shape and the positional information of the phosphene which are necessary for patient's image (subject) perception. Further, by setting the stimulation condition corresponding thereto, the pulse signal converter 102 generates the data for electric stimulation pulse for stimulating the optic nerve from the image information. The thus generated data is transmitted to the internal body device 20 as electromagnetic waves from the transmitter 14. The power from the battery 110 is also transmitted to the internal body device 20 as electromagnetic waves through the transmitter 14. At this time, the information for electric stimulation pulse signal and the power are transmitted in the time-sharing manner. Alternatively, they are transmitted in a state where the power is overlapped with the information for electric stimulation pulse signal.

In the internal body device 20, the data for electric stimulation pulse received by the receiver 21 is input in the information processing control unit 22. The information processing control unit 22 forms the electric stimulation pulse signal from the data for electric stimulation pulse, and controls the electrodes 41a to output the electric stimulation pulse signal. The electric stimulation pulse signal output from the electrodes 41a passes the optic nerve from the optic papilla where the electrodes 41a are inserted, and stimulates the cerebrum. In this manner, the patient perceives the plurality of phosphenes appearing in the visual field, thereby obtaining vision. As described so far, by variously changing the stimulation condition of the electric stimulation pulse signal provided to the optic papilla where nerve fibers distributed all around the retina converge, it is possible to generate phosphenes in a wide range in the patient's vision, whereby the visual perception in a wide visual field can be achieved.

In the embodiment, since the plurality of electrodes 41a are provided in the optic papilla, it is possible to check the generation state of the phosphene under the more various conditions for the patient. Accordingly, it is possible to create more varied patterns of visual perception.

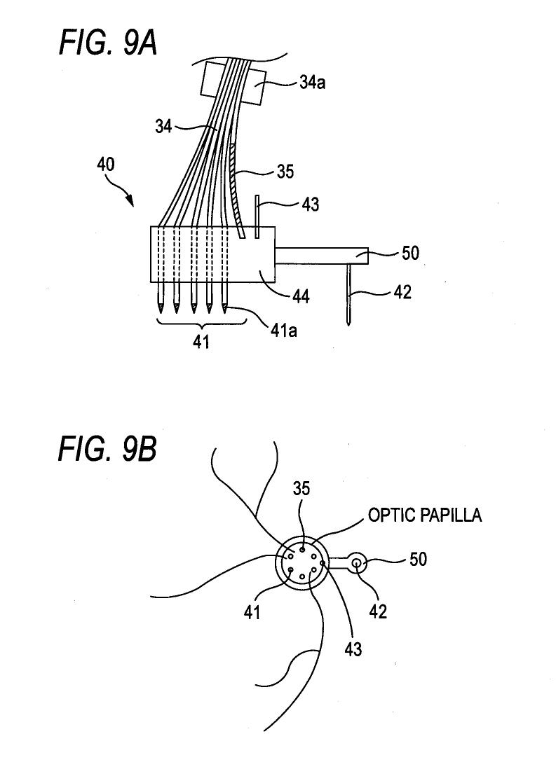

The invention is not limited to the above configuration. FIG. 9 is a view illustrating the configuration of the stimulating unit 40 according to a second embodiment. FIG. 9A is an enlarged view of the stimulating unit 40 of a second embodiment, and FIG. 9B is a view illustrating the position where the stimulating unit 40 is provided in the retina. In FIG. 9, the same configuration as the vision regeneration assisting apparatus 1 is described by using the same figure numbers.

In this case, the fixing portion 42 is provided not in the housing 44 but in a holding portion 50 formed of the same material as the housing 44. The holding portion 50 in which the fixing portion 42 is provided is placed at the position distant from the housing 44 at a certain distance, so that the fixing portion 42 is positioned outside the optic papilla when the electrode portion 41 is positioned on the optic papilla. In this manner, the electrode portion 41 is fixed to the position of the optic papilla without damaging the optic papilla by the fixing portion 42. Although one fixing portion 42 is formed in the holding portion 50 in FIG. 9, it is also possible to form two or more of the fixing portion 42 in the holding portion 50. It is also possible to provide a plurality of holding portions 50 (fixing portion 42) in a direction different from the housing 44. In this manner, it is possible to more stably keep the electrode portion 41 for a long time.

What is claimed is:

1. A vision regeneration assisting apparatus for regenerating a patient's vision, comprising:
a housing;
a plurality of needle-like electrodes that are formed at the housing and extend from the housing to a predetermined length, the electrodes being configured to stick into an optic papilla of a patient's eye; and
a needle-like fixing portion that is formed at the housing, is separated from the electrodes, is formed so that the fixing portion extends from the housing in the same direction in which the electrodes extend, and is configured to maintain a state where the electrodes are stuck in the optic papilla.

2. The vision regeneration assisting apparatus according to claim 1, wherein a part of the fixing portion extending from the housing is longer that parts of the electrodes extending from the housing.

3. The vision regeneration assisting apparatus according to claim 2 comprising a gripping portion for gripping the housing.

4. The vision regeneration assisting apparatus according to claim 3,
wherein the fixing portion and the gripping portion are molded integrally to be a single member.

5. A vision regeneration assisting apparatus for regenerating a patient's vision, comprising:
a plurality of electrodes configured to stick into an optic papilla of a patient's eye;
a controller configured to control the plurality of electrodes to output an electric stimulation pulse signal, respectively; and
a connecting member electrically connecting the electrodes to the controller,
wherein the connecting member includes:
a plurality of first signal wires, each of which is a single wire connected to each of the electrodes;
a plurality of second signal wires, each of which is a stranded wire and includes one end connected to each of the first signal wire and the other end connected to the controller; and
a connecting portion connecting the first signal wire to the second signal wire outside the eyeball.

6. The vision regeneration assisting apparatus according to claim 5, wherein the plurality of second signal wires are bundled and wound in a spiral to form one cable.

7. The vision regeneration assisting apparatus according to claim 6, wherein the first signal wire has a curve shape curved with a radius of curvature of 10 mm or longer and 20 mm or shorter.

8. The vision regeneration assisting apparatus according to claim 7, wherein
the connecting portion is made of a metal pipe,
the first signal wire is welded to and is connected to the second signal wire, and
the first and second signal wires are inserted in the pipe.
